(19) United States
(12) Patent Application Publication
(10) Pub. No.: US 2025/0387329 A1
(43) Pub. Date: Dec. 25, 2025

(54) ORELLANINE FORMULATION

(71) Applicant: ONCORENA AB, Lund (SE)

(72) Inventors: Olivier YAMEOGO, Riom (FR); Emmanuelle COISSARD, Riom (FR); Pal FALCK, Oslo (NO)

(73) Assignee: ONCORENA AB, Lund (SE)

(21) Appl. No.: 18/880,336

(22) PCT Filed: Jun. 23, 2023
(86) PCT No.: PCT/EP2023/067195

§ 371 Date: Dec. 31, 2024

Related U.S. Application Data
(60) Provisional application No. 63/357,757, filed on Jul. 1, 2022.

(51) Int. Cl. A61K 9/08; A61K 9/00; A61K 31/444; A61K 47/02; A61K 47/12

(52) U.S. Cl. CPC A61K 9/08; A61K 9/0019; A61K 31/444; A61K 47/02; A61K 47/12

(57) ABSTRACT

A pharmaceutical aqueous solution formulation comprising Orellanine is disclosed.

### FIELD OF INVENTION

The present invention relates to a pharmaceutical formulation comprising Orellanine.

### BACKGROUND OF THE INVENTION

Orellanine (see Formula I below) is a selective renal toxin occurring in relatively large amounts in several fungal species of the Cortinarius family.

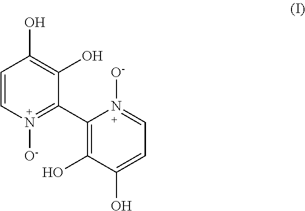

WO 2010/040750 discloses that Orellanine may be used in the treatment of renal cell carcinoma. Orellanine was taken up in human renal cancer cells and killed them with great efficiency whether they were derived from a primary tumor or from metastatic tumor tissue. The cell death progressed for many days after transient exposure to Orellanine, indicating that the toxin was actively taken up and retained by the cells.

Orellanine drug products are being developed as treatments for renal cancer. It is therefore desirable to provide pharmaceutical formulations of Orellanine.

The present inventors have sought to provide stable formulations of Orellanine that are suitable for administration by intravenous injection.

### SUMMARY OF THE INVENTION

Accordingly, the present invention provides a pharmaceutical aqueous solution formulation comprising
from 0.1 to 40 mg/ml of Orellanine; and
a buffer;
wherein the pH of the formulation is in the range of 7 to 8.

The present inventors have found that such formulations are stable and suitable for administration by intravenous injection.

### DETAILED DESCRIPTION OF THE INVENTION

The pharmaceutical formulation is an aqueous formulation. Water for injection (WFI) may be used in aqueous formulations that are to be administered by intravenous injection.

The amount of Orellanine in the formulation is from 0.1 to 40 mg/ml and may be from 0.5 to 30 gm/ml, from 1 to 20 mg/ml of Orellanine, from 5 to 15 mg/ml of Orellanine, from 8 to 12 mg/ml of Orellanine, from 9 to 11 mg/ml of Orellanine, or about 10 mg/ml of Orellanine. In an embodiment, the formulation comprises 10 mg/ml of Orellanine. It is possible to determine the amount of Orellanine in a formulation by conventional methods including High-Performance Liquid Chromatography (HPLC).

The inventors have found that the solubility of Orellanine is pH dependent. Outside the preferred pH range the solubility of Orellanine is low and it is not possible to provide the concentration of Orellanine that is preferred for a pharmaceutical formulation for administration by intravenous injection. The pH of the formulation is in the range of 7 to 8, and may be in the range of 7.1 to 7.6, or in the range of 7.2 to 7.5, or in the range of 7.3 to 7.4. In an embodiment the pH is about 7.4 (from 7.39 to 7.41). pH may be determined by standard methods such as in accordance with Ph. Eur. 2.2.3.

The inventors have found that the concentration of the buffer has an impact on the solubility of Orellanine. The buffer concentration may be from 10 to 200 mM, or from 20 to 180 mM, or from 30 to 120 mM, or from 40 to 80 mM, or from 40 to 60 mM.

The buffer may be a phosphate buffer such as NaH₂PO₄—Na₂HPO₄ or Citrate-Na₂HPO₄.

The buffer ensures that the pH is within the chosen range. Further adjustment of the pH may be carried out by addition of base or acid, such as addition of sodium hydroxide or hydrochloric acid, e.g. with sodium hydroxide or hydrochloric acid at 0.2M.

The inventors have found that the osmolality of the formulation affects the solubility of the Orellanine. The osmolality of the formulation may be greater than 200 mOsm, or may be in the range of from 250 to 350 mOsm. Osmolality may be determined by standard methods, such as in accordance with Ph. Eur. 2.2.35. In one embodiment of the invention, the osmolality of the formulation is such that it is within the isotonic range, i.e. the formulation has an isotonicity that is similar to the isotonicity of blood.

The ionic strength of the formulation may be increased by the addition of sodium chloride. The formulation may comprise from 0.05 to 2 wt % sodium chloride (wherein the weight is based upon the weight of the formulation), or from 0.1 to 1 wt % sodium chloride, or from 0.5 to 0.9 wt % sodium chloride, or about 0.8 wt % sodium chloride.

The inventors have found that by controlling the pH and the osmolality it is possible to prepare stable formulations wherein the concentration of Orellanine is appropriate for intravenous administration.

In an embodiment, the pharmaceutical aqueous solution formulation comprises from 0.1 to 40 mg/ml of Orellanine; a phosphate buffer wherein the concentration of the buffer is from 10 to 200 mM; and from 0.1 to 1 wt % sodium chloride; wherein the pH of the formulation is in the range of 7 to 8 and optionally wherein the osmolality of the formulation is greater than 200 mOsm.

In another embodiment, the pharmaceutical aqueous solution formulation comprises from 1 to 20 mg/ml of Orellanine; a phosphate buffer wherein the concentration of the buffer is from 10 to 200 mM; and from 0.1 to 1 wt % sodium chloride; wherein the pH of the formulation is in the range of 7 to 8 and optionally wherein the osmolality of the formulation is greater than 200 mOsm.

In another embodiment, the pharmaceutical aqueous solution formulation comprises from 5 to 15 mg/ml of Orellanine; a phosphate buffer wherein the concentration of the buffer is from 10 to 200 mM; and from 0.1 to 1 wt % sodium chloride; wherein the pH of the formulation is in the range of 7 to 8 and optionally wherein the osmolality of the formulation is greater than 200 mOsm.

In another embodiment, the pharmaceutical aqueous solution formulation comprises from 0.1 to 40 mg/ml of Orellanine; a phosphate buffer chosen from NaH₂PO₄—Na₂HPO₄ or Citrate-Na₂HPO₄ wherein the concentration of the buffer is from 10 to 200 mM; and from 0.1 to 1 wt % sodium chloride; wherein the pH of the formulation is in the range of 7 to 8 and optionally wherein the osmolality of the formulation is greater than 200 mOsm.

In another embodiment, the pharmaceutical aqueous solution formulation comprises from 1 to 20 mg/ml of Orellanine; a phosphate buffer chosen from NaH₂PO₄—Na₂HPO₄ or Citrate-Na₂HPO₄ wherein the concentration of the buffer is from 10 to 200 mM; and from 0.1 to 1 wt % sodium chloride; wherein the pH of the formulation is in the range of 7 to 8 and optionally wherein the osmolality of the formulation is greater than 200 mOsm.

In another embodiment, the pharmaceutical aqueous solution formulation comprises from 5 to 15 mg/ml of Orellanine; a phosphate buffer chosen from NaH₂PO₄—Na₂HPO₄ or Citrate-Na₂HPO₄ wherein the concentration of the buffer is from 10 to 200 mM; and from 0.1 to 1 wt % sodium chloride; wherein the pH of the formulation is in the range of 7 to 8 and optionally wherein the osmolality of the formulation is greater than 200 mOsm.

In another embodiment, the pharmaceutical aqueous solution formulation comprises from 0.1 to 40 mg/ml of Orellanine; a phosphate buffer wherein the concentration of the buffer is from 40 to 60 mM; and from 0.5 to 0.9 wt % sodium chloride; wherein the pH of the formulation is in the range of 7.2 to 7.5 and optionally wherein the osmolality of the formulation is in the range of from 250 to 350 mOsm.

In another embodiment, the pharmaceutical aqueous solution formulation comprises from 1 to 20 mg/ml of Orellanine; a phosphate buffer wherein the concentration of the buffer is from 40 to 60 mM; and from 0.5 to 0.9 wt % sodium chloride; wherein the pH of the formulation is in the range of 7.2 to 7.5 and optionally wherein the osmolality of the formulation is in the range of from 250 to 350 mOsm.

In another embodiment, the pharmaceutical aqueous solution formulation comprises from 5 to 15 mg/ml of Orellanine; a phosphate buffer wherein the concentration of the buffer is from 40 to 60 mM; and from 0.5 to 0.9 wt % sodium chloride; wherein the pH of the formulation is in the range of 7.2 to 7.5 and optionally wherein the osmolality of the formulation is in the range of from 250 to 350 mOsm.

In another embodiment, the pharmaceutical aqueous solution formulation comprises from 0.1 to 40 mg/ml of Orellanine; a phosphate buffer chosen from NaH₂PO₄—Na₂HPO₄ or Citrate-Na₂HPO₄ wherein the concentration of the buffer is from 40 to 60 mM; and from 0.5 to 0.9 wt % sodium chloride; wherein the pH of the formulation is in the range of 7.2 to 7.5 and optionally wherein the osmolality of the formulation is in the range of from 250 to 350 mOsm.

In another embodiment, the pharmaceutical aqueous solution formulation comprises from 1 to 20 mg/ml of Orellanine; a phosphate buffer chosen from NaH₂PO₄—Na₂HPO₄ or Citrate-Na₂HPO₄ wherein the concentration of the buffer is from 40 to 60 mM; and from 0.5 to 0.9 wt % sodium chloride; wherein the pH of the formulation is in the range of 7.2 to 7.5 and optionally wherein the osmolality of the formulation is in the range of from 250 to 350 mOsm.

In another embodiment, the pharmaceutical aqueous solution formulation comprises from 5 to 15 mg/ml of Orellanine; a phosphate buffer chosen from NaH₂PO₄—Na₂HPO₄ or Citrate-Na₂HPO₄ wherein the concentration of the buffer is from 40 to 60 mM; and from 0.5 to 0.9 wt % sodium chloride; wherein the pH of the formulation is in the range of 7.2 to 7.5 and optionally wherein the osmolality of the formulation is in the range of from 250 to 350 mOsm.

The formulation may be prepared by a method comprising steps of:
a) adding a buffer to an aqueous solution of Orellanine;
b) stirring; and
c) adjusting the pH by addition of additional buffer, by addition of base (e.g. sodium hydroxide), and/or by addition of acid (e.g. hydrochloride acid).

Alternatively, the formulation may be prepared by a method comprising steps of:
a) adding Orellanine (optionally as an aqueous solution) to a buffer;
b) stirring; and
c) adjusting the pH by addition of additional buffer, by addition of base (e.g. sodium hydroxide), and/or by addition of acid (e.g. hydrochloride acid).

The steps of stirring and adjusting the pH may be repeated several times to ensure that the final pH is within the chosen range. The inventors have observed that the pH of the formulation may change upon stirring such that pH adjustment is needed to provide a pH within the chosen range and to provide a stable formulation.

The buffer may be prepared by combining the buffer components (e.g. as NaH₂PO₄ and Na₂HPO₄, or sodium citrate and Na₂HPO₄) with water for injection and with sodium chloride.

The formulation may be filtered (e.g. through a 0.22 μm PVDF filter) before it is packaged. The formulation may be packaged in conventional vessels, such as glass injection vials. The vials may be stoppered and sealed with flip off caps.

The formulation may be used in a method of treating cancer such as renal cancer. The formulation may be used for the treatment of patients suffering from or susceptible to renal cell carcinoma. The treatment may be used to treat a tumor that is localized to one or both of the patient's kidneys. Alternatively, the renal cell carcinoma may have metastasized such that at least one renal cell carcinoma tumor is present in at least one non-kidney tissue.

The formulation is suitable for administration by intravenous injection.

The formulation may be administered in a single dose, in a series of daily doses, or in an intermittent dosing format (e.g. a plurality of doses or dose sequences administered between 1 and about 30 days apart, between 1 and about 14 days apart, or between 1 and about 7 days apart).

When administered in a single dose, a dose of between about 1 mg/kg and about 100 mg/kg of Orellanine may be administered to the patient, or a dose of between about 2 mg/kg and about 25 mg/kg of Orellanine, or a dose of between about 5 mg/kg and about 15 mg/kg of Orellanine.

The formulation may be administered in two or more doses. The doses may be administered daily or intermittently (e.g. with at least one non-administration day separating sequential doses). When administered in a plurality of doses, a dose of between about 0.5 mg/kg and about 10 mg/kg of Orellanine may be administered to the patient, or a dose of between about 1 mg/kg and 5 mg/kg, or a dose of about 2 mg/kg.

If sequential doses are administered intermittently, the sequential doses may be administered between two and seven days apart. The formulation may be administered to the patient in three, four, five or six or more doses and wherein each dose is administered between three and five days apart. The formulation may be administered to the patient in four, five, or six or more doses administered between three and four days apart, wherein each dose comprises between about 1 mg/kg to about 20 mg/kg of Orellanine, or about 2 to about 10 mg/kg of Orellanine, or about 5 mg/kg of Orellanine.

A daily dose of the formulation may be administered for at least two days. Typical daily doses administered to patients are between 0.1 and 10 mg/kg, or between 1 and 5 mg/kg, or about 2 mg/kg. Therapeutic protocols may comprise daily administration of the formulation between 5 and about 30 days, or preferably between 10 and 20 days, or most preferably about 14 days.

In certain instances, it may be desirable to conduct a plurality of intermittent administration protocols, daily administration protocols, or a combination thereof, as described above, in combination with rest and/or recovery periods. Thus, in certain instances, it may be desirable to administer the formulation according to a daily or intermittent administration method, measure the tumor response to the therapy, and then conduct subsequent daily or intermittent administration therapies as necessary to eliminate or further reduce the size of the renal cancer tumors. Such administration strategies are well known to the person with normal skills in the field of oncology.

In one embodiment of the present invention a patient suffering from renal cell carcinoma may be treated with the formulation by daily injections of about 0.5-5 mg Orellanine/kg b.w., most preferably about 2 mg Orellanine/kg b.w., for about 7-21 consecutive days, or about 14 consecutive days. One to 5 hours after each daily injection of the formulation, most preferably about 2 hours after such injection, the patient is subjected to hemodialysis for 1-5 h, most preferably about 2 h, in order to eliminate any Orellanine that has not been taken up into tumor tissue and thereby minimize any undesired side effects that might occur in the extracellular space.

The preferred doses and dose regimes described above are based on a human being weighing 70 kg and suffering from renal cell carcinoma with a tumor burden of about 1 kg. However, as is readily known to the worker with normal skills in the field of cancer medicine, such preferred doses and dose regimes are governed to a large extent by patient characteristics such as age, sex, weight, general condition and, above all, the individual patient's tumor burden and response to the treatment. As always, the ultimate responsibility for choosing the proper dose and treatment strategy lies with the physician in charge of the patient.

The invention will now be described by reference to examples which are not intended to be limiting of the invention:

### Examples

Solubility Study with Buffer (pH Dependent Solubility Testing)

A phase solubility study was performed for different buffers to analyse the effect of pH on the solubility of Orellanine. Four buffers were tested:
Acetate buffer pH 4.5 at 50 mM-prepared from 1.81 g sodium acetate and 14 ml of acetic acid (2N), adjusted up to 11 with water for injection (WFI).
Phosphate buffer pH 5.8 at 50 mM-prepared from 92 ml of NaH₂PO₄ and 8 ml of Na₂HPO₄, adjusted to 200 ml with WFI.
Phosphate buffer pH 6.8 at 50 mM-prepared from 51 ml of NaH₂PO₄ and 49 ml of Na₂HPO₄, adjusted to 200 ml with WFI.
Phosphate buffer pH 7.4 at 50 mM-prepared from 19 ml of NaH₂PO₄ and 81 ml of Na₂HPO₄, adjusted to 200 ml with WFI.

The pH of each buffer was adjusted with a pH meter and sodium hydroxide or hydrochloric acid at 0.2M.

The solubility of Orellanine in each buffer was tested by weighing 40 mg of Orellanine into a vial, adding 2 ml of buffer, stirring the suspension for 1 hour, and then stirring at 400 rpm for 24 hours at room temperature. Each suspension was centrifuged at 5300 rpm for 15 minutes. The supernatant was filtered with a 0.45 μm nylon filter. Table 1 shows the outcome of the testing:

TABLE 1

| | | | Osmolality | Concentration of |
|---|---|---|---|---|
| | Appearance | | of | Orellanine in |
| | of | | supernatant | supernatant |
| | supernatant | pH | (mOsm) | (mg/ml) |
| Acetate buffer pH | Colourless | 4.66 | 119 | 0.0024 |
| 4.5 at 50 mM | solution | | | |
| Phosphate buffer | Colourless | 5.84 | 102 | 0.0299 |
| pH 5.8 at 50 mM | solution | | | |
| Phosphate buffer | Yellowish | 6.79 | 119 | 0.6218 |
| pH 6.8 at 50 mM | solution | | | |
| Phosphate buffer | Yellowish | 7.08 | 135 | 1.7150 |
| pH 7.4 at 50 mM | solution | | | |

The maximum concentration of Orellanine achieved was 1.7 mg/ml with phosphate buffer pH 7.4 at 50 mM.

Solubility Study with Different Buffer Concentrations at pH 7.4

The impact of buffer concentration was assessed. Three buffers were used:
Phosphate buffer pH 7.4 at 25 mM-prepared from 0.95 ml of NaH₂PO₄ (0.1M) and 4.05 ml of Na₂HPO₄ (0.1M), adjusted to 20 ml with WFI.
Phosphate buffer pH 7.4 at 50 mM-prepared from 19 ml of NaH₂PO₄ (0.1M) and 81 ml of Na₂HPO₄ (0.1M), adjusted to 200 ml with WFI.
Phosphate buffer pH 7.4 at 100 mM-prepared from 38 ml NaH₂PO₄ (0.1M) and 162 ml of Na₂HPO₄ (0.1M), adjusted to 200 ml with WFI.

The pH of each buffer was adjusted with a pH meter and sodium hydroxide or hydrochloric acid at 0.2M.

The solubility of Orellanine in each buffer was tested by weighing 20 mg of Orellanine into a 5 ml vial, adding 2 ml of buffer, stirring the suspension for 3 hours, and then adjusting the pH to 7.4 with sodium hydroxide or hydrochloric acid at 0.2M. The suspension was then stirred at 400 rpm for 24 hours at room temperature. Each suspension was filtered through a 0.2 μm PVDF syringe filter. Table 2 shows the outcome of the testing:

TABLE 2

| | Appearance | | | | Concentration | |
|---|---|---|---|---|---|---|
| | after 24 hours | Appearance | | Osmolality | of Orellanine | Purity of |
| | of stirring | after filtration | pH | (mOsm) | (mg/ml) | Orellanine |
| NaH₂PO₄—Na₂HPO₄ | Yellow | Yellow | 7.29 | 92 | 3.68 | 99.3 |
| pH 7.4 25 mM | solution with | solution | | | | |
| | particles in | | | | | |
| | suspension | | | | | |
| NaH₂PO₄—Na₂HPO₄ | Yellow | Yellow | 7.33 | 150 | 4.88 | 100 |
| pH 7.4 50 mM | solution | solution | | | | |
| NaH₂PO₄—Na₂HPO₄ | Yellow | Yellow | 7.30 | 242 | 4.91 | 99.4 |
| pH 7.4 100 mM | solution | solution | | | | |

The solubility ranged from 3.7 to 4.9 mg/ml for these formulations. 50 mM seems to be the optimum concentration of buffer from a solubility and purity perspective.

Solubility Study with Different Buffer Salts at pH 7.4

Four buffers at pH 7.4 and 50 mM were assessed:
Phosphate buffer pH 7.4 at 50 mM-prepared from 19 ml of NaH₂PO₄ (0.1M) and 81 ml of Na₂HPO₄ (0.1M), adjusted to 200 ml with WFI.
Phosphate buffer pH 7.4 at 50 mM-prepared from 28.1 ml of KH₂PO₄ (0.2M) and 21.9 ml of NaOH (0.2M) adjusted to 200 ml with WFI.
Tris-HCl buffer pH 7.4 at 50 mM-prepared from 54.7 ml of tris(hydroxymethyl)aminomethane (0.1M) and 45.3 ml of hydrochloric acid (0.1M), adjusted to 200 ml with WFI.
Citrate phosphate buffer pH 7.4 at 50 mM-prepared from 2.4 ml of citric acid monohydrate (0.2M) and 47.6 ml of Na₂HPO₄ (0.2M), adjusted to 200 ml with WFI.

The pH of each buffer was adjusted with a pH meter and sodium hydroxide or hydrochloric acid at 0.2M.

The solubility of Orellanine in each buffer was tested by weighing 20 mg of Orellanine into a 5 ml vial, adding 2 ml of buffer, stirring the suspension for 3 hours, and then adjusting the pH to 7.4 with sodium hydroxide or hydrochloric acid at 0.2M. The suspension was then stirred at 400 rpm for 24 hours at room temperature. Each suspension was filtered through a 0.2 μm PVDF syringe filter. Table 3 shows the outcome of the testing:

TABLE 3

| | Appearance | Appearance | | | Concentration | |
|---|---|---|---|---|---|---|
| | after 24 hours | after | | Osmolality | of Orellanine | Purity of |
| | of stirring | filtration | pH | (mOsm) | (mg/ml) | Orellanine |
| NaH₂PO₄—Na₂HPO₄ | Yellow | Yellow | 7.33 | 150 | 4.88 | 100 |
| pH 7.4 | solution | solution | | | | |
| KH₂PO₄—NaOH | Yellow | Yellow | 7.40 | 108 | 4.56 | 99.4 |
| pH 7.4 | solution | solution | | | | |
| Tris-HCl pH 7.4 | Yellow | Yellow | 7.49 | 95 | 4.59 | 99.5 |
| Citrate - Na₂HPO₄ | Yellow | Yellow | 7.32 | 157 | 5.44 | 99.5 |
| pH 7.4 | solution | solution | | | | |

The best solubility was provided by the NaH₂PO₄—Na₂HPO₄ and Citrate - Na₂HPO₄ buffers.

Solubility Study with Different Ionic Strengths at pH 7.4

The ionic strength of the NaH₂PO₄—Na₂HPO₄ buffer was adjusted by adding sodium chloride. The ionic strength of the NaH₂PO₄—Na₂HPO₄ buffer at 50 mM was estimated at around 130 mM. The proportion of sodium chloride in added to the buffers was 0.41%, 0.98%, and 2.15%.

The solubility of Orellanine in each buffer was tested by weighing 20 mg of Orellanine into a 5 ml vial, adding 2 ml of buffer, stirring the suspension for 3 hours, and then adjusting the pH to 7.4 with sodium hydroxide or hydrochloric acid at 0.2M. The suspension was then stirred at 400 rpm for 24 hours at room temperature. Each suspension was filtered through a 0.2 μm PVDF syringe filter. Table 4 shows the outcome of the testing:

TABLE 4

| | Appearance | | | | Concentration | |
|---|---|---|---|---|---|---|
| | after 24 hours | Appearance | | Osmolality | of Orellanine | Purity of |
| | of stirring | after filtration | pH | (mOsm) | (mg/ml) | Orellanine |
| NaH₂PO₄—Na₂HPO₄ | Yellow | Yellow | 7.26 | 205 | 5.31 | 99.4 |
| pH 7.4 at 50 mM plus | solution | solution | | | | |
| 0.41% NaCl | | | | | | |
| NaH₂PO₄—Na₂HPO₄ | Yellow | Yellow | 7.19 | 328 | 5.90 | 99.4 |
| pH 7.4 at 50 mM plus | solution | solution | | | | |
| 0.98% NaCl | | | | | | |
| NaH₂PO₄—Na₂HPO₄ | Yellow | Yellow | 7.30 | 595 | 6.43 | 99.4 |
| pH 7.4 at 50 mM plus | solution | solution | | | | |
| 2.15% NaCl | | | | | | |

Increasing the ionic strength increased the osmolality of the solution and promoted Orellanine solubility.

### Formulation Adjustment

Two buffers were selected: NaH₂PO₄—Na₂HPO₄ pH 7.4 and Citrate-Na₂HPO₄ PH 7.4 at 50 mM, each with addition of 0.70% sodium chloride. Adding 0.70% sodium chloride provides a solution close to isotonicity.

Formulations were prepared by weighing 40 mg of Orellanine into a 5 ml vial, adding 2 ml of buffer, stirring the suspension for 1 hour, adjusting the pH to 7.4 with sodium hydroxide (0.2M), stirring the suspension at 400 rpm for 24 hours at room temperature, and adjusting the pH to 7.4 with sodium hydroxide (0.2M). Each suspension was filtered through a 0.2 μm PVDF syringe filter. Table 5 shows the properties of the formulations:

TABLE 5

| | Appearance | | | | Concentration | |
|---|---|---|---|---|---|---|
| | after 24 hours | Appearance | | Osmolality | of Orellanine | Purity of |
| | of stirring | after filtration | pH | (mOsm) | (mg/ml) | Orellanine |
| NaH₂PO₄—Na₂HPO₄ | White to | Yellow | 7.29 | 312 | 11.1 | 99.5 |
| pH 7.4 at 50 mM plus | off-white | solution | | | | |
| 0.70% NaCl | suspension | | | | | |
| Citrate - Na₂HPO₄ | White to | Yellow | 7.24 | 315 | 10.5 | 99.4 |
| pH 7.4 at 50 mM plus | off-white | solution | | | | |
| 0.70% NaCl | suspension | | | | | |

Solubility of greater than 10 mg/ml was achieved by twice adjusting the pH to 7.4: firstly after 1 hour of stirring and again after an overnight stirring.

1. A pharmaceutical aqueous solution formulation comprising from 0.1 to 40 mg/ml of Orellanine; and a buffer; wherein the pH of the formulation is in the range of 7 to 8.

2. The formulation according to claim 1, comprising from 1 to 20 mg/ml of Orellanine.

3. The formulation according to claim 2, comprising from 5 to 15 mg/ml of Orellanine.

4. The formulation according to claim 1, wherein the pH of the formulation is in the range of 7.1 to 7.6.

5. The formulation according to claim 4, wherein the pH of the formulation is in the range of 7.2 to 7.5.

6. The formulation according to claim 1, wherein the concentration of the buffer is from 10 to 200 mM.

7. The formulation according to claim 6, wherein the concentration of the buffer is from 40 to 60 mM.

8. The formulation according to claim 1, wherein the buffer is a phosphate buffer.

9. The formulation according to claim 8, wherein the buffer is NaH2PO4-Na2HPO4 or Citrate-Na2HPO4.

10. The formulation according to claim 1, wherein osmolality of the formulation is greater than 200 mOsm.

11. The formulation according to claim 10, wherein the osmolality of the formulation is in the range of from 250 to 350 mOsm.

12. The formulation according to claim 1, comprising sodium chloride.

13. The formulation according to claim 12, comprising from 0.1 to 1 wt % sodium chloride.

14. (canceled)

15. (canceled)

16. (canceled)

17. A method for preparing a formulation according to claim 1, comprising steps of:
adding a buffer to an aqueous solution of Orellanine, or adding Orellanine, optionally as an aqueous solution, to a buffer;
stirring; and
adjusting the pH by addition of additional buffer, by addition of base (e.g. sodium hydroxide), and/or by addition of acid (e.g. hydrochloride acid).

18. A method according to claim 17, wherein steps (b) and (c) are carried out at least twice.

19. A method for treating cancer, wherein the method comprises intravenously administering a formulation according to claim 1 to a subject in need thereof.

20. The method of claim 19, comprising providing the formulation to a subject in need thereof for at least two days, wherein the daily dose is between 0.1 and 10 mg/kg of body weight.

21. A kit for intravenous administration, comprising the formulation according to claim 1 packaged in a container, and optionally further comprising information for providing the formulation to a subject in need thereof.

22. The method of claim 19, wherein the cancer is renal cancer.
